(19) United States
(12) Patent Application Publication
(10) Pub. No.: US 2023/0162869 A1
(43) Pub. Date: May 25, 2023

(54) Error Correction in Measurements of Medical Parameters

(71) Applicant: ChroniSense Medical Ltd., Yokneam (IL)

(72) Inventor: Daniel H. Lange, Kfar Vradim (IL)

(21) Appl. No.: 17/532,966

(22) Filed: Nov. 22, 2021

(51) Int. Cl. G16H 50/70; G16H 40/40; G16H 10/60; G16H 50/20

(52) U.S. Cl. CPC G16H 50/70; G16H 40/40; G16H 10/60; G16H 50/20

(57) ABSTRACT

Systems and methods for error correction in measurements of medical parameters are provided. An example method includes storing, in a database, a plurality of records, where a record of the plurality records includes a calibration parameter and a set of measurement errors corresponding to a set of values of a medical parameter of the user. The method also includes distributing the plurality of records to a plurality of groups and receiving a further record. The further record includes a further set of further measurement errors corresponding to a further set of further values of the medical parameter of a further user. The method includes selecting based on the further record, a group of the plurality of groups and determining, based on records belonging to the group, a further calibration parameter for correcting results of measurements of the medical parameter of the further user.

### FIELD

The present application relates to systems and methods for monitoring health status of people, and, more specifically, to systems and methods for error correction in measurements of medical parameters of people.

### BACKGROUND

It should not be assumed that any of the approaches described in this section qualify as prior art merely by virtue of their inclusion in this section.

Constant monitoring of basic medical parameters of people can facilitate diagnosing a human health condition and early prediction of chronic illnesses. Nowadays, the medical parameters can be monitored constantly using various wearable devices, such as activity trackers and smart watches. However, most of the wearable devices measure the medical parameters indirectly. For example, blood pressure and oxygen saturation can be measured using optical sensors. However, indirect measurements strongly depend on individual physiological variables of patients. Accordingly, the indirect measurements can lead to errors in values of medical parameters. Therefore, the wearable devices are needed to be calibrated to account for measurement errors using more accurate measurement devices, such as cuff-based medical devices and catheter-based medical device. Typically, calibration of such devices requires multiple simultaneous measurements by a wearable device and more accurate measurement devices, which can be inconvenient or not even practical. Thus, there is a need for more convenient techniques of calibration of the wearable devices.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

According to one aspect of the present disclosure, a system for error correction in measurements of medical parameters is provided. The system may include a database storing a plurality of records, where a record of the plurality records includes at least one calibration parameter and a set of measurement errors corresponding to a set of values of at least one medical parameter of the user. The system may also include a processor configured to distribute the plurality of records to a plurality of groups. The processer may receive a further record including a further set of further measurement errors corresponding to a further set of further values of the medical parameter of a further user. Upon receipt of the further record, the processor may select, based on the further record, a group of the plurality of groups. The processor may then determine, based on records associated with the group, at least one further calibration parameter. The further calibration parameter can be used to correct a result of measurement of the medical parameter of the further user.

The records can be distributed based on a similarity measure. The similarity measure may define a distance between first measurement errors of a first record in the plurality of records and second measurement errors of a second record in the plurality of records.

The processor can be configured to receive the further record from a user measurement device associated with the further user and provide the at further calibration parameter to the user measurement device. The user measurement device can be configured to measure a new value of the at least one medical parameter associated with the further user, and correct the new value using the further calibration parameter.

The set of values of medical parameters of the user can be measured by a user measurement device. The set of errors can be determined based on a set of reference values for the medical parameter of the user. The reference values can correspond to the values. The reference values for the medical parameter of the user can be measured by a reference measurement device substantially simultaneously to corresponding values of one medical parameter of the user. The reference measurement device may have a higher measurement accuracy than the user measurement device. The calibration parameter can be determined based on the set of reference values and the set of values using a learning model. The calibration parameter can be used by the user measurement device to correct results of measurements of the medical parameter of the user.

The values in the set of values of the medical parameter of the user can be determined at pre-defined times within a pre-determined time interval. The values of the set of values of the medical parameter can be results of indirect measurements of parameters such as: an oxygen saturation, a respiratory rate, a blood pressure, and blood glucose level.

The record in the database may also include a value of at least one of the following characteristics of the user: an age and a gender. The further record may include a further value of the at least one characteristics of the further user.

According to another example embodiment of the present disclosure, a method for error correction in measurements of medical parameters is provided. The method may include storing, by a processor, in a database, a plurality of records, where a record of the plurality records includes at least one calibration parameter and a set of measurement errors corresponding to a set of values of at least one medical parameter of the user. The method may also include distributing, by the processor, the plurality of records to a plurality of groups. The method may include receiving, by the processor, a further record including a further set of further measurement errors corresponding to a further set of further values of the medical parameter of a further user. The method may include selecting, by the processor and based on the further record, a group of the plurality of groups. The method may also include determining, by the processor and based on records belonging to the group, at least one further calibration parameter for correcting a result of measurement of the at least one medical parameter of the further user.

According to another example embodiment of the present disclosure, the steps of the method for error correction in measurements of medical parameters are stored on a non-transitory machine-readable medium comprising instructions, which when implemented by one or more processors perform the recited steps.

Other example embodiments of the disclosure and aspects will become apparent from the following description taken in conjunction with the following drawings.

### BRIEF DESCRIPTION OF THE DRAWINGS

Embodiments are illustrated by way of example and not limitation in the figures of the accompanying drawings, in which like references indicate similar elements.

FIG. 1 is a block diagram showing an example environment, wherein a method for error correction in measurements of medical parameters can be implemented.

FIG. 2 is a block diagram showing components of an example user measurement device.

FIG. 3 is block diagram illustrating calibration of a user measurement device.

FIG. 4 shows an example plot of measurement errors of a systolic blood pressure, according to an example embodiment.

FIG. 5 shows an example multidimensional plot of records including measurements errors of values of a medical parameter.

FIG. 6 is a flow chart showing an example method for error correction in measurements of medical parameters.

FIG. 7 shows a diagrammatic representation of a computing device for a machine, within which a set of instructions for causing the machine to perform any one or more of the methodologies discussed herein can be executed.

### DETAILED DESCRIPTION

The following detailed description includes references to the accompanying drawings, which form a part of the detailed description. The drawings show illustrations in accordance with exemplary embodiments. These exemplary embodiments, which are also referred to herein as “examples,” are described in enough detail to enable those skilled in the art to practice the present subject matter. The embodiments can be combined, other embodiments can be utilized, or structural, logical and electrical changes can be made without departing from the scope of what is claimed. The following detailed description is, therefore, not to be taken in a limiting sense, and the scope is defined by the appended claims and their equivalents.

The present disclosure provides systems and methods for error correction in measurements of medical parameters of users. Specifically, embodiments of the present disclosure may facilitate calibration process of user measurements devices that are configured to measure the medical parameters in an indirect manner. Certain embodiment of the present disclosure may provide determination of calibration parameters individual to a particular user, a particular medical parameter, and a particular type of user measurement device. Some embodiments may help to significantly decrease the period of the calibration process.

As used herein, indirectly measured parameters (also referred to herein as unobserved parameters or derived parameters) are parameters derived or calculated from directly measured parameters (also referred to herein as observed parameters). Examples of directly measured parameters are pulse rate and pulse transit time. The directly measured parameters can be derived directly from measured signals, for example a photoplethysmography (PPG) signal or an electrocardiography (ECG) signal. The PPG signal can be measured using an optical sensor. The ECG signal can be measured using an ECG sensor. The pulse rate can be calculated directly by counting peaks in the PPG signal. The pulse transit time can be also calculated directly by counting a time difference between the peaks of the PPG signal and peaks of the QRS peaks in ECG signal.

The indirectly measured parameters can be calculated from the directly measured parameters using estimation formulas or models. Examples of the indirectly measured parameters are blood oxygen saturation, respiratory rate, blood pressure, blood glucose level, and so forth. The estimation formulas and models for determining the indirectly measured parameters may include calibration parameters can be calibrated to account for individual physiological variables of a patient, such as individual cardiovascular characteristics, skin tone, anatomical details, elasticity of blood vessels, and so on.

Some of the existing models for determining medical parameters can reasonably propagate information from observed parameters to unobserved parameters via physiological modeling and calibration. However, traditional calibration is often highly inefficient and typically results in biased solutions.

Unlike existing calibration techniques, embodiments of the present disclosure provide a framework that uses global mapping between observed parameters and unobserved parameters. Specifically, embodiments of the present disclosure allow grouping individual patients according to characteristic errors in derived parameters of the patients. The mapping can be based on big data databases and result in determining scaling curves (also referred herein to as user-specific error curves) for correcting individual derived parameters. The mapping may allow discovering scaling curves that have not been previously demonstrated in existing technologies for calculating or correcting derived medical parameters. Implementing scaling curves derived from big data may facilitate achieving better performance and generalizability for correcting indirectly measured parameters than currently available with oversimplified analytical models.

While some embodiments of the present disclosure are described with reference to determination of scaling curves for systolic blood pressure, methods of the present disclosure can be applied to determination of scaling curves for any indirectly measured medical parameters. It should be noted that the embodiments of the present disclosure can be also used for determining scaling curves for directly measured medical parameters.

According to some example embodiments, a system for error correction in measurements of medical parameters may include a database for storing a plurality of records, where a record of the plurality records includes at least one calibration parameter and a set of measurement errors corresponding to a set of values of at least one medical parameter of the user. The system may include a processor configured to distribute the plurality of records to a plurality of groups. The processer may receive a further record including a further set of further measurement errors corresponding to a further set of further values of the medical parameter of a further user. The processor may select, based on the further record, a group of the plurality of groups. The processor may then determine, based on records belonging to the group, at least one further calibration parameter. The further calibration parameter can be used to correct a result of measurement of the medical parameter of the further user.

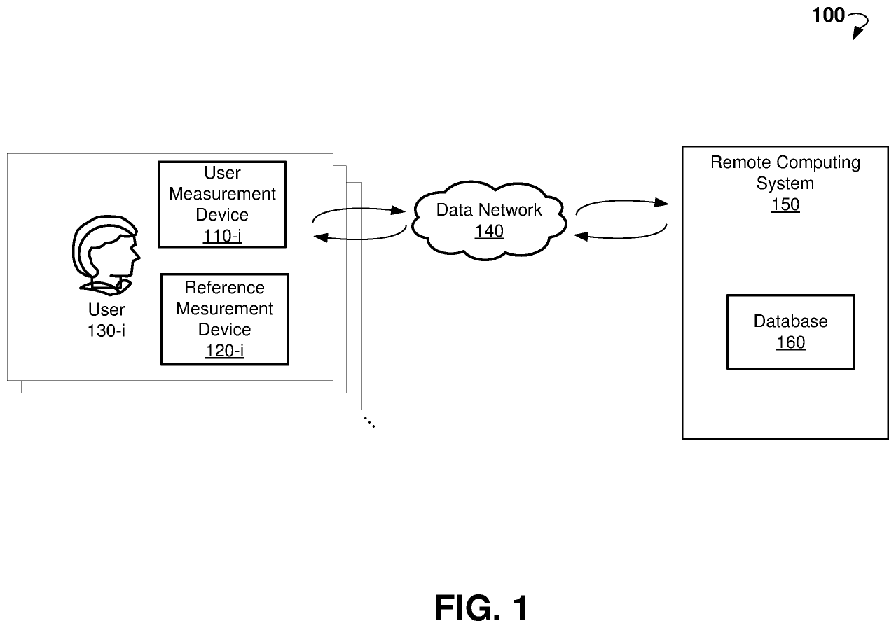

Referring now to FIG. 1, an example environment 100 is provided. The environment 100 can be used to implement methods for error correction in measurements of medical parameters. The environment 100 can include one or more user measurement devices 110-i, one or more reference measurement devices 120-i, a data network 140, and a remote computing system 150.

In some embodiments, the user measurement device 110-i can be worn by a user 130-i, for example, on a wrist, ankle, earlobe, neck, chest, fingertip, and the like, for an extended period of time. The user measurement device 110-i can be carried out as a watch, a bracelet, a wristband, a belt, a neck band, and the like. The user measurement device 110-i can be configured to measure, for the user 130-i, values of one or more medical parameters in a non-intrusive manner while, for example, the patient is at home, at work, outdoors, traveling, or is located at some other stationary or mobile environment. The medical parameters may include a blood pressure, a blood glucose level, pulse rate, respiratory rate, pulse transition time, oxygen saturation, and so forth.

The reference measurement device 120-i can be used by the user 130-i to measure reference values of the one or more medical parameters. The reference measurement device 120-i may measure the one or more medical parameters with a higher accuracy than the user measurement device 110-i. For example, the user measurement device 110-i can measure blood pressure of the user 130-i using an optical sensor. The reference measurement device 130-i can include a cuff-based blood pressure measurement device utilizing an inflatable cuff to pressurize a blood artery. In further embodiments, the reference measurement device can include a professional medical device operable by a medical professional, where the blood pressure measurements involve insertion of a catheter into a human artery.

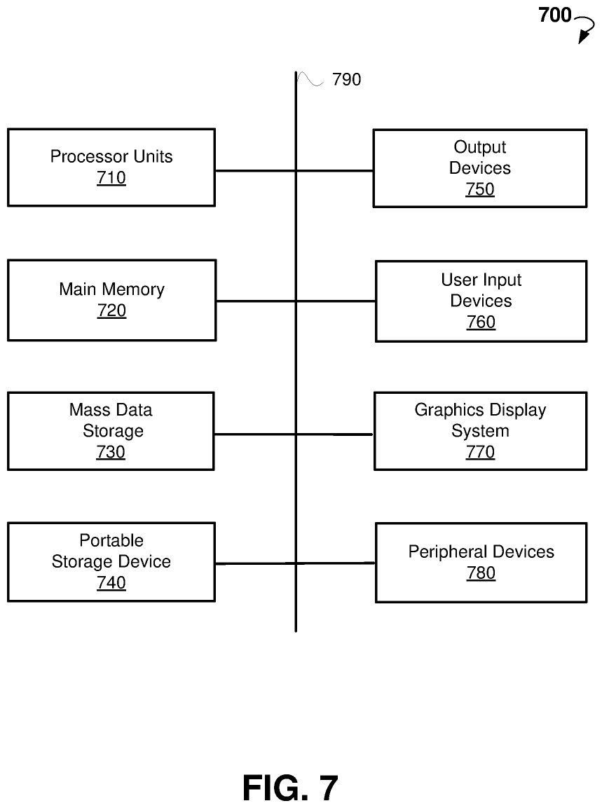

The remote computing system 150 may include a cloud-based computing resource (also referred to as a cloud). In some embodiments, the remote computing system 150 may include one or more server farms/clusters comprising a collection of computer servers and is co-located with network switches and/or routers. An example remote computing system is described in more detail below with reference to FIG. 7.

The remote computing system 150 may store a database 160 of records. Each of the records in database 160 may include a set of measurement errors of one or more medical parameters. To obtain the measurement errors, the user 130-i may simultaneously measure the medical parameters by the user measurement device 110-i and the reference measurement device 120-i. The user may enter, via a user interface, the results of the measurements by the reference measurement device 120-i into the user measurement device 110-i. The user measurement device 110-i may determine the measurement errors as a difference between the results of measurement of the medical parameters by the reference measurement device 120-i and the user measurement device 110-i.

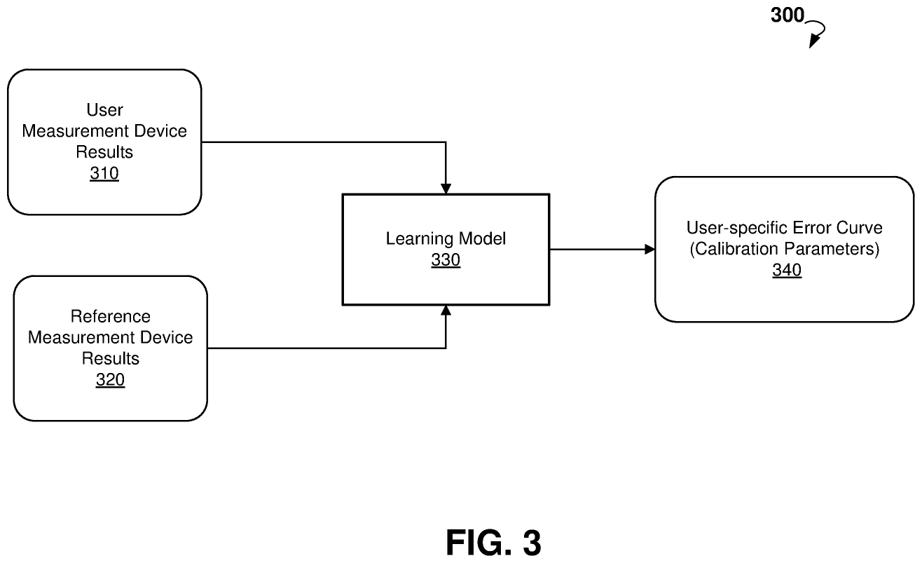

The measurement errors can be provided to the remote computing system 150 via the data network 140. The remote computing system 150 may further determine a calibration parameter for correcting results of further measurements of the user measurement device 110-i by two techniques described in more detail below with reference to FIGS. 3, 4, and 5. The remote computing system 150 can send the calibration parameter to the user measurement device 110-i.

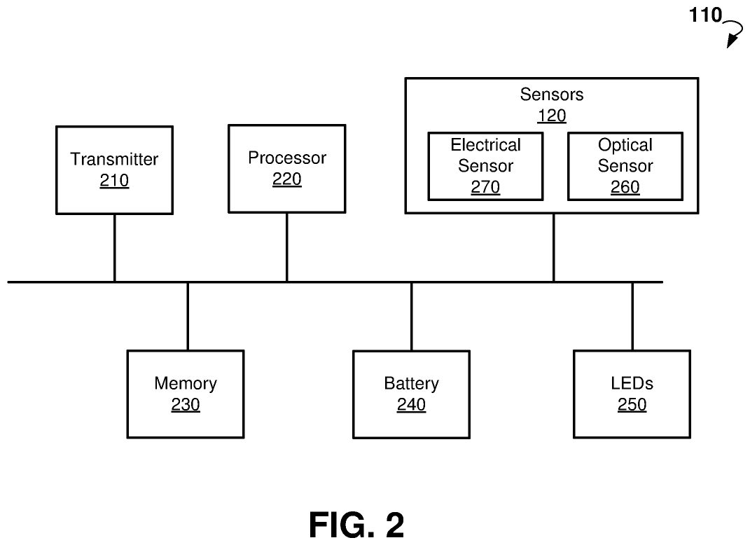

FIG. 2 is a block diagram illustrating components of user measurement device 110, according to an example embodiment. The example user measurement device 110 may include a transmitter 210, a processor 220, memory storage 230, a battery 240, light-emitting diodes (LEDs) 250, optical sensor 260, and electrical sensor 270. The user measurement device 110 may comprise additional or different components to provide a particular operation or functionality. Similarly, in other embodiments, the user measurement device 110 includes fewer components that perform similar or equivalent functions to those depicted in FIG. 2.

The transmitter 210 can be configured to communicate with a network such as the Internet, a Wide Area Network (WAN), a Local Area Network (LAN), a cellular network, and so forth, to send data streams, for example measurement errors of medical parameters.

The processor 220 can include hardware and/or software, which is operable to execute computer programs stored in memory 230. The processor 220 can use floating point operations, complex operations, and other operations, including processing and analyzing data obtained from electrical sensor 270 and optical sensor 260.

In some embodiments, the battery 240 is operable to provide electrical power for operation of other components of user measurement device 110. In some embodiments, the battery 240 is a rechargeable battery. In certain embodiments, the battery 240 is recharged using an inductive charging technology.

In various embodiments, the LEDs 250 are operable to emit light signals. The light signals can be of a red wavelength (typically 660 nm) or infrared wavelength (660 nm). Each of the LEDs is activated separately and accompanied by a “dark” period where neither of the LEDs is on to obtain ambient light levels. In some embodiments, a single LED can be used to emit both the infrared and red light signals. The lights can be absorbed by human blood (mostly by hemoglobin). The oxygenated hemoglobin absorbs more infrared light while deoxygenated hemoglobin absorbs more red light. Oxygenated hemoglobin allows more red light to pass through while deoxygenated hemoglobin allows more infrared light to pass through. In some embodiments of the present disclosure, the LEDs 250 are also operable to emit light signals of isosbestic wavelengths (typically 810 nm and 520 nm). Both oxygenated hemoglobin and deoxygenated hemoglobin absorb the light of the isosbestic wavelengths equally.

The optical sensor(s) 260 (typically a photodiode) can receive light signals modulated by human tissue. Intensity of the modulated light signal represents a PPG. Based on the changes in the intensities of the modulated light signals, one or more medical parameters, such as, for example, oxygen saturation, arterial blood flow, pulse rate, and respiration, can be determined.

The LEDs 250 and optical sensor(s) 260 can be utilized in either a transmission or a reflectance mode for pulse oximetry. In the transmission mode, the LEDs 250 and sensor 260 are typically attached or clipped to a translucent body part (e.g., a finger, toe, and earlobe). The LEDs 250 are located on one side of the body part while the optical sensor(s) 260 are located directly on the opposite site. The light passes through the entirety of the body part, from one side to the other, and is thus modulated by the pulsating arterial blood flow. In the reflectance mode, the LEDs 250 and optical sensor(s) 260 are located on the same side of the body part (e.g. a forehead, a finger, and a wrist), and the light is reflected from the skin and underlying near-surface tissues back to the optical sensor(s) 260.

FIG. 3 is block diagram illustrating calibration of a user measurement device, according to some example embodiment. In some embodiments, the calibration can be performed by the learning model 330. In certain embodiments, the learning model 330 can be implemented as instructions stored in a memory of the user measurement device 110-i and executable by a processor of the user measurement device 110-i. In other embodiments, the learning model 330 can be implemented as an application running on the remote computer system 150.

The learning model 330 can receive results 310 of measurements of one or more medical parameters performed by user measurement device 110-i and results 320 of measurements of the same one or more medical parameters performed strenuously by reference measurement device 110-i.

To collect the results 310 and 320, the user 130-i can be prompted to perform measurements by user measurement device 110-i and reference measurement device 110-i at different times within a pre-determined time interval. For example, the user can be prompted to perform the measurements after sleep, before going to bed, before eating, after eating, before performing physical exercises, and after performing physical exercise, and so forth. The purpose of collecting the results of measurements is to receive values (v₁, v₂, . . . , v_{N}) of a medical parameter measured by the user measurement device 110-i and reference values (r₁, r₂, . . . , r_{N}) of a medical parameter measured by the reference measurement device 120-i, such that: 1) the value vᵢ is measured substantially at the same time as corresponding reference value rᵢ, for i=1, . . . , N; 2) the range of values (v₁, v₂, . . . , v_{N}) and reference values (r₁, r₂, . . . , r_{N}) are distributed densely over a possible range of the medical parameter; 3) number N of the values is sufficient to obtain calibration parameters by the learning model 330.

The learning model 330 may determine, based on the values (v₁, v₂, . . . , v_{N}) and reference values (r₁, r₂, . . . , r_{N}), a user-specific error curve E(v, p₁, . . . , p_{L}). The error curve E(v, p₁, . . . , p_{L}) returns an error estimate for a value v of medical parameter measured by user measurement device 110-i. The pᵢ, . . . , p_{L} are calibration parameters specific to the user 130-i and determined by the learning model 330. The learning model 330 may include various models, such as regression, neural network, and so forth.

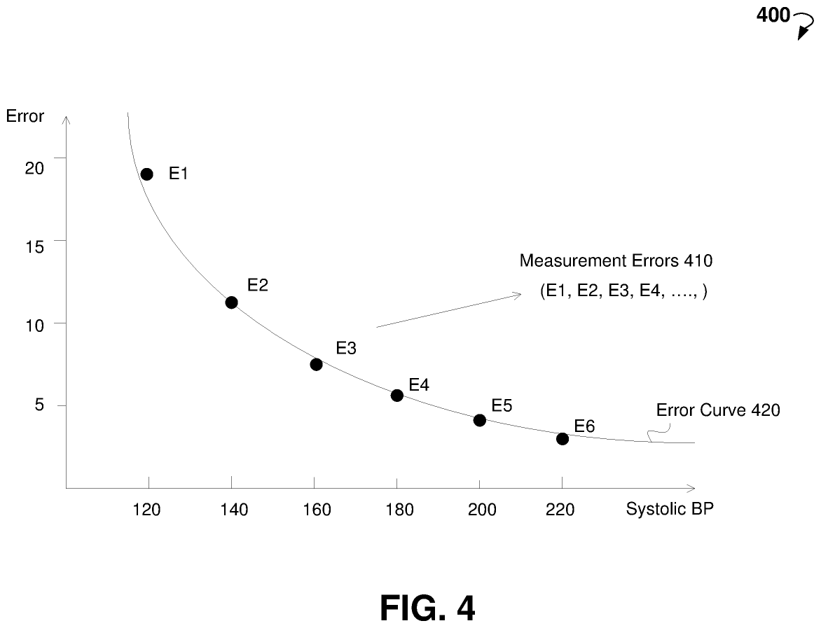

FIG. 4 shows an example plot 400 of a measurement errors of a systolic blood pressure, according to an example embodiment. The measurements errors 410 are differences between reference values (r₁, r₂, . . . , r_{N}) of systolic blood pressure measured by reference measurement device 120-i and values (v₁, v₂, . . . , v_{N}) systolic blood pressure measured by user measurement device 110-i simultaneously with the reference measurement device 120-i. The values cover the range of systolic blood pressure. The curve 420 shows the error curve E(v, p₁, . . . , p_{L}) determined based on reference values (r₁, r₂, . . . , r_{N}) and values (v₁, v₂, . . . , v_{N}).

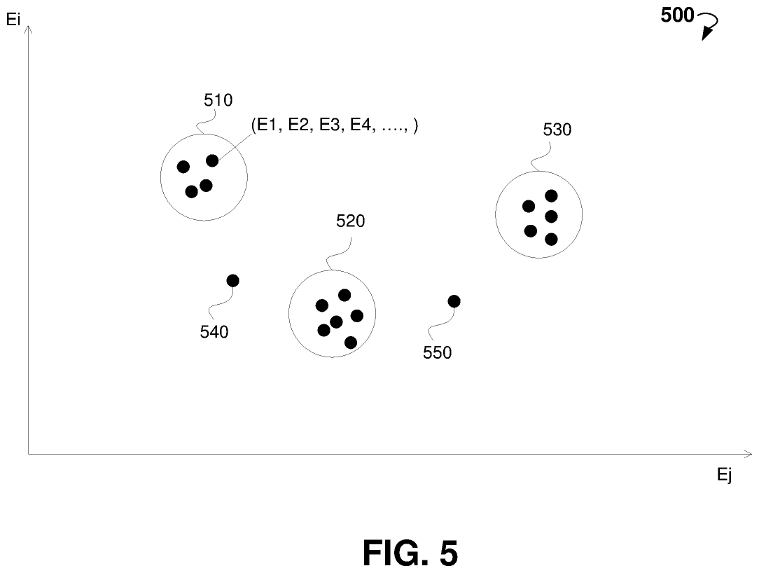

FIG. 5 shows an example multidimensional plot 500 of records including measurements errors of values of a medical parameter of a user. The records can be stored in the database 160. Each of the records may include measurements errors of values of more than one medical parameters. For example, a record may include measurements errors for values of blood pressure, and measurements errors for values of oxygen saturation, and measurement levels for blood glucose level. The record may also include one of characteristics of a user, for example age or gender. Some of the records may also include calibration parameters corresponding to the measurement errors.

The records can be distributed in groups based on a similarity measure. The similarity measure can be a distance between the records as points in a multidimensional space. In example of FIG. 1, the records are distributed in groups 510, 520, and 530. At the same time, the database 160 may include records, such as records 540 and 550 in FIG. 5, that cannot be referred to any of the groups 510, 520, and 530.

According to some embodiments of the present disclosure, a user measurement device 110-k of a new user can collect a new record including measurement errors of values of a medical parameter of the new user. Prior to determining calibration parameters for the new user via the learning model 330, the user measurement device 110-k may send the new record to the remote computing system 150.

The remote computing system 150 may select a group from the groups 510, 520, and 530, such that the group includes records having measurements errors similar to the new record. Assuming that the records in the selected group include calibration parameters that were previously determined based on the measurement errors in the records, the remote computing system 150 may determine a new calibration parameter based on the calibration parameters corresponding to the records in the selected group.

In other embodiments, the remote computing system 150 may select a single record in the database 160 that includes measurement errors similar (based on the similarity measure) to measurement errors in the new record. If the selected record includes a calibration parameter previously determined based on the measurement errors of the record, then the new record (new user) can be assigned the same calibration parameter.

The new calibration parameter can be sent to the user measurement device 110-k of the new user. The user measurement device 110-k can use the new calibration parameter to correct results of new measurements of medical parameters of the new user. Alternatively, the user measurement device 110-k can use the new calibration parameter as an initial guess in the learning model 330. In both these cases, it will take the new user less time to calibrate the user measurement device 110-k.

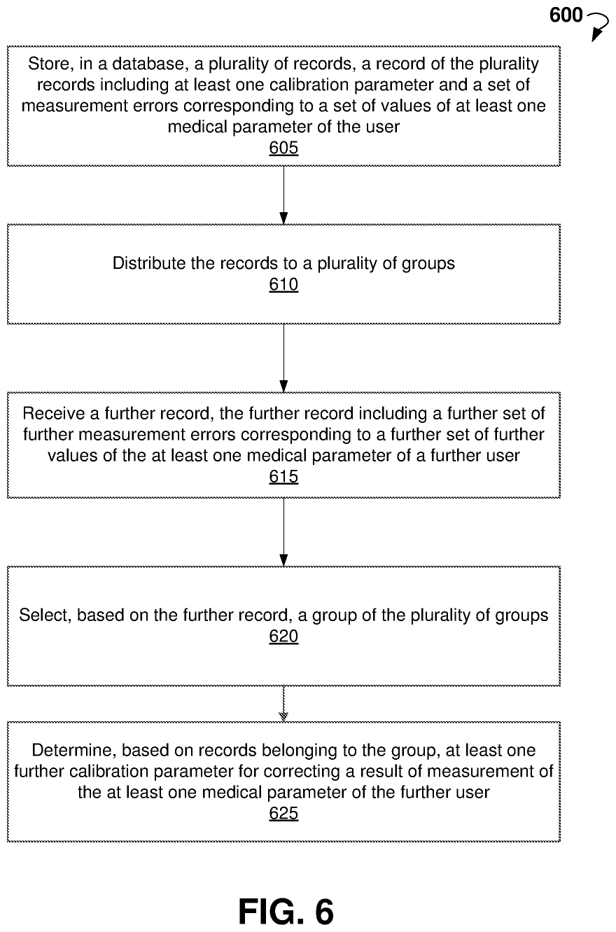

FIG. 6 is a flow chart showing steps of a method 600 for error correction in measurements of medical parameters, according to some embodiments. The method 600 can be implemented using remote computer system 150 in environment 100 described in FIG. 1.

The method 600 may commence in block 602 with storing, by a processor, to a database, a plurality of records. A record of the plurality records may include at least one calibration parameter and a set of measurement errors corresponding to a set of values of at least one medical parameter of the user. The set of values of medical parameter of the user can be measured by a user measurement device associated with the user. The set of errors can be determined based on a set of reference values for the medical parameter of the user corresponding to the values. The reference values for the medical parameter of the user can be measured by a reference measurement device substantially simultaneously with measurement of corresponding values for one medical parameter of the user. The reference measurement device may have a higher measurement accuracy than the user measurement device.

The calibration parameter can be determined based on the set of reference values and the set of values using a learning model. The user measurement device may use the calibration parameter to correct results of measurements of the medical parameters of the user.

The values in the set of values of the medical parameter of the user can be determined at pre-defined times within a pre-determined time interval. The values of the set of values of the medical parameter can be results of indirect measurements of one of the following: an oxygen saturation, a respiratory rate, a blood pressure, and a blood glucose level.

In block 610, the method 600 may proceed with distributing, by the processor, the plurality of records to a plurality of groups. The records can be distributed based on a similarity measure. The similarity measure can define a distance between first measurement errors of a first record in the plurality of records and a second measurements errors of a second record in the plurality of records.

In block 615, the method 600 may proceed with receiving, by the processor, a further record. The further record may include a further set of further measurement errors corresponding to a further set of further values of the medical parameter of a further user. The further record can be received from a user measurement device associated with the further user.

In block 620, the method 600 may proceed with selecting, by the processor and based on the further record, a group of the plurality of groups. The selected group may include records distanced with respect to the further record by a pre-determined threshold.

In block 625, the method 600 may include determining, by the processor and based on records belonging to the group, at least one further calibration parameter to be used to correct results of measurement of the at least one medical parameter of the further user. The further calibration parameters can be determined based on the calibration parameters of the records. The further calibration parameters can be provided to the user measurement device. The user measurement device can measure a new value of the medical parameter for the further user. The user measurement device can correct the new value using the further calibration parameter.

FIG. 7 illustrates a computer system 700 that may be used to implement embodiments of the present disclosure, according to an example embodiment. The computer system 700 may serve as a computing device for a machine, within which a set of instructions for causing the machine to perform any one or more of the methodologies discussed herein can be executed. The computer system 700 can be implemented in the contexts of the likes of computing systems, networks, servers, or combinations thereof. The computer system 700 includes one or more processor units 710 and main memory 720. Main memory 720 stores, in part, instructions and data for execution by processor units 710. Main memory 720 stores the executable code when in operation. The computer system 700 further includes a mass data storage 730, a portable storage device 740, output devices 750, user input devices 760, a graphics display system 770, and peripheral devices 780. The methods may be implemented in software that is cloud-based.

The components shown in FIG. 7 are depicted as being connected via a single bus 790. The components may be connected through one or more data transport means. Processor units 710 and main memory 720 are connected via a local microprocessor bus, and mass data storage 730, peripheral devices 780, the portable storage device 740, and graphics display system 770 are connected via one or more I/O buses.

Mass data storage 730, which can be implemented with a magnetic disk drive, solid state drive, or an optical disk drive, is a non-volatile storage device for storing data and instructions for use by processor units 710. Mass data storage 730 stores the system software for implementing embodiments of the present disclosure for purposes of loading that software into main memory 720.

The portable storage device 740 operates in conjunction with a portable non-volatile storage medium, such as a floppy disk, compact disk (CD), Digital Versatile Disc (DVD), or USB storage device, to input and output data and code to and from the computer system 700. The system software for implementing embodiments of the present disclosure is stored on such a portable medium and input to the computer system 700 via the portable storage device 740.

User input devices 760 provide a portion of a user interface. User input devices 760 include one or more microphones, an alphanumeric keypad, such as a keyboard, for inputting alphanumeric and other information, or a pointing device, such as a mouse, a trackball, stylus, or cursor direction keys. User input devices 760 can also include a touchscreen. Additionally, the computer system 700 includes output devices 750. Suitable output devices include speakers, printers, network interfaces, and monitors.

Graphics display system 770 includes a liquid crystal display or other suitable display device. Graphics display system 770 receives textual and graphical information and processes the information for output to the display device. Peripheral devices 780 may include any type of computer support device to add additional functionality to the computer system.

The components provided in the computer system 700 of FIG. 7 are those typically found in computer systems that may be suitable for use with embodiments of the present disclosure and are intended to represent a broad category of such computer components that are well known in the art. Thus, the computer system 700 can be a personal computer, handheld computing system, telephone, mobile computing system, workstation, tablet, phablet, mobile phone, server, minicomputer, mainframe computer, or any other computing system. The computer may also include different bus configurations, networked platforms, multi-processor platforms, and the like. Various operating systems may be used including UNIX, LINUX, WINDOWS, MAC OS, PALM OS, ANDROID, IOS, QNX, TIZEN and other suitable operating systems.

It is noteworthy that any hardware platform suitable for performing the processing described herein is suitable for use with the embodiments provided herein. Computer-readable storage media refer to any medium or media that participate in providing instructions to a central processing unit, a processor, a microcontroller, or the like. Such media may take forms including, but not limited to, non-volatile and volatile media such as optical or magnetic disks and dynamic memory, respectively. Common forms of computer-readable storage media include a floppy disk, a flexible disk, a hard disk, magnetic tape, any other magnetic storage medium, a CD Read Only Memory disk, DVD, Blu-ray disc, any other optical storage medium, RAM, Programmable Read-Only Memory, Erasable Programmable Read-Only Memory, Electronically Erasable Programmable Read-Only Memory, flash memory, and/or any other memory chip, module, or cartridge.

In some embodiments, the computer system 700 may be implemented as a cloud-based computing environment, such as a virtual machine operating within a computing cloud. In other embodiments, the computer system 700 may itself include a cloud-based computing environment, where the functionalities of the computer system 700 are executed in a distributed fashion. Thus, the computer system 700, when configured as a computing cloud, may include pluralities of computing devices in various forms, as will be described in greater detail below.

In general, a cloud-based computing environment is a resource that typically combines the computational power of a large grouping of processors (such as within web servers) and/or that combines the storage capacity of a large grouping of computer memories or storage devices. Systems that provide cloud-based resources may be utilized exclusively by their owners or such systems may be accessible to outside users who deploy applications within the computing infrastructure to obtain the benefit of large computational or storage resources.

The cloud may be formed, for example, by a network of web servers that comprise a plurality of computing devices, such as the computer system 700, with each server (or at least a plurality thereof) providing processor and/or storage resources. These servers may manage workloads provided by multiple users (e.g., cloud resource customers or other users). Typically, each user places workload demands upon the cloud that vary in real-time, sometimes dramatically. The nature and extent of these variations typically depends on the type of business associated with the user.

Thus, methods and systems for error correction in measurements of medical parameters have been described. Although embodiments have been described with reference to specific example embodiments, it will be evident that various modifications and changes can be made to these example embodiments without departing from the broader spirit and scope of the present application. Accordingly, the specification and drawings are to be regarded in an illustrative rather than a restrictive sense.

What is claimed is:

1. A system for error correction in measurements of medical parameters, the system comprising:
a database storing a plurality of records, a record of the plurality records including at least one calibration parameter and a set of measurement errors corresponding to a set of values of at least one medical parameter of the user; and
a processor configured to:
distribute the plurality of records to a plurality of groups;
receive a further record, the further record including a further set of further measurement errors corresponding to a further set of further values of the at least one medical parameter of a further user;
select, based on the further record, a group of the plurality of groups; and
determine, based on records belonging to the group, at least one further calibration parameter to be used to correct a result of measurement of the at least one medical parameter of the further user.

2. The system of claim 1, wherein the records are distributed based on a similarity measure, the similarity measure defining a distance between first measurement errors of a first record in the plurality of records and second measurement errors of a second record in the plurality of records.

3. The system of claim 1, wherein the processor is configured to:
receive the further record from a user measurement device, the user measurement device being associated with the further user; and
provide the at least one further calibration parameter to the user measurement device.

4. The system of claim 3, wherein the user measurement device is configured to:
measure, for the further user, a new value of the at least one medical parameter; and
correct the new value using the at least one further calibration parameter.

5. The system of claim 1, wherein:
the set of values of at least one medical parameter of the user is measured by a user measurement device associated with the user; and
the set of errors is determined based on a set of reference values for the at least one medical parameter of the user, the reference values corresponding to the values.

6. The system of claim 4, wherein the reference values for the at least one medical parameter of the user are measured by a reference measurement device substantially simultaneously to corresponding values for the at least one medical parameter of the user, the reference measurement device having a higher measurement accuracy than the user measurement device.

7. The system of claim 4, wherein the at least one calibration parameter is:
determined based on the set of reference values and the set of values using a learning model; and
used by the user measurement device to correct results of measurements of the at least one medical parameter of the user.

8. The system of claim 1, wherein values in the set of values of the at least one medical parameter of the user are determined at pre-determined times within a pre-determined time interval.

9. The system of claim 1, wherein the values of the set of values of the at least one medical parameter are results of indirect measurement of one of the following: an oxygen saturation, a respiratory rate, a blood pressure, and a blood glucose level.

10. The system of claim 1, wherein:
the record includes a value of at least one of the following characteristics of the user: an age and a gender; and
the further record includes a further value of the at least one characteristics of the further user.

11. A method for error correction in measurements of medical parameters, the method comprising:
storing, by a processor in a database, a plurality of records, a record of the plurality records including at least one calibration parameter and a set of measurement errors corresponding to a set of values of at least one medical parameter of the user;
distributing, by the processor, the plurality of records to a plurality of groups;
receiving, by the processor, a further record, the further record including a further set of further measurement errors corresponding to a further set of further values of the at least one medical parameter of a further user;
selecting, by the processor and based on the further record, a group of the plurality of groups; and
determining, by the processor and based on records belonging to the group, at least one further calibration parameter to be used to correct a result of measurement of the at least one medical parameter of the further user.

12. The method of claim 11, wherein the records are distributed based on a similarity measure, the similarity measure defining a distance between first measurement errors of a first record in the plurality of records and a second measurements errors of a second record in the plurality of records.

13. The method of claim 11, wherein the further record is received from a user measurement device, the user measurement device being associated with the further user, and further comprising, providing the at least one further calibration parameter to the user measurement device.

14. The method of claim 13, further comprising:
measuring, by the user measurement device for the further user, a new value of the at least one medical parameter; and
correcting, by the user measurement device, the new value using the at least one further calibration parameter.

15. The method of claim 11, wherein:
the set of values of at least one medical parameter of the user is measured by a user measurement device associated with the user; and
the set of errors is determined based on a set of reference values for the at least one medical parameter of the user, the reference values corresponding to the values.

16. The method of claim 14, wherein the reference values for the at least one medical parameter of the user are measured by a reference measurement device substantially simultaneously to corresponding values for the at least one medical parameter of the user, the reference measurement device having a higher measurement accuracy than the user measurement device.

17. The method of claim 14, wherein the at least one calibration parameter is:
determined based on the set of reference values and the set of values using a learning model; and
used by the user measurement device to correct results of measurements of the at least one medical parameters of the user.

18. The method of claim 11, wherein values in the set of values of the at least one medical parameter of the user are determined at pre-determined times within a pre-determined time interval.

19. The method of claim 11, wherein the values of the set of values of the at least one medical parameter are results of indirect measurement of one of the following: an oxygen saturation, a blood pressure, and a blood glucose level.

20. A non-transitory computer-readable storage medium having embodied thereon instructions, which when executed by at least one processor, perform steps of a method, the method comprising:
storing, in a database, a plurality of records, a record of the plurality records including at least one calibration parameter and a set of measurement errors corresponding to a set of values of at least one medical parameter of the user;
distributing the plurality of records to a plurality of groups;
receiving a further record, the further record including a further set of further measurement errors corresponding to a further set of further values of the at least one medical parameter of a further user;
selecting based on the further record, a group of the plurality of groups; and
determining, based on records belonging to the group, at least one further calibration parameter to be used to correct a result of measurement of the at least one medical parameter of the further user.
